(19) United States
(12) Patent Application Publication
(10) Pub. No.: US 2004/0022740 A1
(43) Pub. Date: Feb. 5, 2004

(54) Macrolide formulations for inhalation and methods of treatment of endobronchial infections

(76) Inventors: William R. Baker, Bellevue, WA (US); Peter B. Challoner, Seattle, WA (US); Ribhi M. Shawar, Bellevue, WA (US); Kay K. Huh, Emeryville, CA (US); David M. Ryckman, Bellevue, WA (US)

Correspondence Address: Chiron Corporation, Intellectual Property R440, PO Box 8097, Emeryville, CA, 94662-8097, US

(21) Appl. No.: 10/333,948

(22) PCT Filed: Jul. 10, 2001
(86) PCT No.: PCT/US01/41328

(51) Int. Cl. A61K 31/70; A61K 31/7048; A61L 9/04

(52) U.S. Cl. 424/45; 514/28; 514/29; 128/200.23

(57) ABSTRACT

Macrolide formulations, such as an erythromycylamine formulation, for delivery by aerosolization are described. The concentrated erythromycylamine formulations contain an amount of erythromycylamine effective to treat infections caused by susceptible bacteria. Unit dose devices having a container containing a formulation of the macrolide antibiotic in a physiologically acceptable carrier are also described. Methods for treatment of pulmonary infections by a formulation (liquid solution, suspension, or dry powder) delivered as an aerosol having mass median aerodynamic diameter predominantly between 1 to 5 μm are also described.

### FIELD OF THE INVENTION

This invention concerns novel and improved macrolide formulations, such as erythromycylamine formulations, for delivery by inhalation and to improved methods of treatment of susceptible acute or chronic endobronchial infections. In particular, the invention relates to formulations comprising at least one concentrated macrolide antibiotic in a physiologically acceptable liquid solution or dry powder form. The formulations are suitable for delivery of a macrolide antibiotic drug, such as erythromycylamine, to the lung endobronchial airway space of a liquid aerosol or dry powder aerosol form, wherein a substantial portion of the aerosolized droplets or particles of the formulation have a mass median aerodynamic diameter between 1 to 5 μm. Formulated and aerosol delivered efficacious amounts of the macrolides are effective for the treatment and/or prophylaxis of acute and chronic endobronchial infections, and pneumonia, particularly those caused by Streptococcus pneumoniae, Haemophilus influenzae, Staphylococcus aureus, Moraxella catarrhalis, Legionella pneumophila, Chlamydia pneumoniae, and Mycoplasma pneumoniae. The novel formulations have small volume yet deliver an effective dose of the macrolide antibiotic to the site of infection. In yet other aspects, this invention relates to new and improved unit dose formulations of macrolide antibiotics for delivery by aerosol inhalation.

### BACKGROUND OF THE INVENTION

Streptococcus pneumonia and other typical and atypical pathogens infect the endobronchial space in the lung of individuals who suffer from chronic obstructive pulmonary disease (COPD) [S. Chodosh et al. Clinical Infectious Diseases 1998; 27: 730-738]. COPD is most commonly manifested as chronic bronchitis (CB) and emphysema.

Chronic bronchitis is a pulmonary disease that is characterized by the inflammation and progressive destruction of lung tissue. The debilitation of the lungs in CB patients is associated with chronic cough, increased daily sputum production, and accumulation of purulent sputum produced as a result of chronic endobronchial infections caused by compromised pulmonary function. Acute exacerbation of chronic bronchitis (AECB) is often characterized by increasing cough, purulent sputum production, and clinical deterioration caused by Streptococcus pneumonia, H. influenzae, and Moraxella catarrhalis. Pneumonia may also result from infection by these organisms either de novo or as a complication of COPD. Despite the controversy over the appropriateness of antimicrobacterial therapy for the treatment of CB and in particular acute exacerbations of CB, Saint et al. (JAMA 1995; 273: 957-960) demonstrated that oral antimicrobial therapy provided some clinical benefit when compared to no therapy. Furthermore, the dose of antimicrobial agent was important with respect to time to relapse. Thus, higher doses of oral antimicrobial agents were associated with a higher median infection free-interval (S. Chodosh et al., Clinical Infectious Diseases 1998; 27: 730-738).

Presently, oral administration of macrolides and fluoroquinolones active against typical and atypical pathogens are treatments of choice for CB. However, oral administration of macrolide antibiotics has adverse side effects. The most common side effects associated with the treatment of oral/parental macrolide antibiotics are diarrhea/loose stools, nausea, abdominal pain and vomiting (R. N. Brogden D. Peters, Drugs, 1994; 48: 599-616 and H. D. Langtry, R. N. Brogden Drugs 1997; 53: 973-1004 and references cited therein). In addition, pseudomembranous colitis is a serious side effect associated with oral antibiotic therapy including oral macrolide therapy (S. H. Ahmad et al. Indian J. Pediatr. 1993, 60: 591-594). Penetration of macrolides into lung tissue after oral administration varies according to dose and composition (R. N. Brogden D. Peters, Drugs, 1994; 48: 599-616 and H. D. Langtry, R. N. Brogden Drugs 1997; 53: 973-1004 and references cited therein). Furthermore, macrolides are associated with alterations in the systemic concentrations of unrelated drugs, such as theophylline, due to interactions with the cytochrome-based metabolic system of the liver. Such drug-drug interactions often require dosage adjustment or elimination of one component from treatment regimes.

Erythromycylamine is a 14-membered ring macrolide belonging to the erythromycin family of antibiotics and possesses a similar in vitro antibiotic spectrum to erythromycin A, and like erythromycin A, is an effective treatment of typical and atypical pneumonias. Erythromycylamine has a C-9 amino function having the S-configuration in place of the C-9 carbonyl group found in erythromycin A. One significant limitation of erythromycylamine is its lack of oral absorption, thus, in order to achieve useful therapeutic concentrations a prodrug, dirithromycin, was developed. The prodrug of erythromycylamine is dirithromycin, which features a bridged acetal function between the C-9 amino and C-11 hydroxy groups (see FIG. 1). The cyclic acetal is rapidly hydrolysized in plasma by a nonenzymatic process (half-life of approximately 30 minutes). Dirithromycin has been shown to successfully treat exacerbations that occur in patients with CB (M. Cazzola et al., Respiratory Medicine; 1998; 92: 895-901). A major advantage of erythromycylamine is its long half-life (30-44 hours) (R. N. Brogden D. Peters, Drugs, 1994; 48: 599-616). Unfortunately, oral bioavailability of dirithromycin is only 10-14% in humans with high elimination (62-81%) into the feces mostly as erythromycylamine. Because erythromycylamine is not absorbed and its prodrug, dirithromycin, is poorly absorbed, limited amounts of active drug substance are available systemically to treat lung infections caused by typical and atypical bacteria. While enough erythromycylamine concentrates at the site of infection to provide a therapeutic effect, the concentration of drug is limited. Higher oral doses or more frequent dosing of dirithromycin increase drug concentration at the site of action; however, increased adverse events are likely to occur and may increase patient hardship and compliance.

One of the first studies using aerosolized antibiotics for the treatment of lung infections was reported in Lancet, 22:1377-9 (1981). A controlled, double-blind study on twenty CF patients demonstrated that aerosol administration of carbenicillin and the aminoglycoside gentamicin can improve the health of CF patients. Since that time, scattered reports in the literature have examined aerosol delivery of aminoglycosides in general and tobramycin in particular (see, for example, U.S. Pat. No. 5,580,269). However, evaluation and comparison of these studies is often difficult because of the differences in antibiotic formulations, breathing techniques, nebulizers and compressors. Moreover, aerosol delivery is often difficult to evaluate because of differences in the formulations, aerosol delivery devices, dosages, particle sizes, regimens, and the like. When, for example, the mass median aerodynamic diameter (MMAD) is greater than 5 μm, the particles are typically deposited in the upper airways, decreasing the amount of antibiotic delivered to the site of infection in the lower respiratory tract. An article published in Arch. Dis. Child., 68:788 (1993) emphasized the need for standardized procedures and for improvement in aerosol administration of drugs to CF patients.

Effective aerosol administration is currently compromised by the lack of additive-free and physiologically compatible formulations and particularly by the inability of certain nebulizers to generate small and uniform particle sizes. The size range of aerosolized particles needed to deliver the drug to the endobronchial space and peripheral lung, the sites of the infection is preferably between about 1 and 5 μm. Many nebulizers that aerosolize therapeutics, including aminoglycosides, produce a large number of aerosol particles having sizes less than 1 μm or greater than 5 μm. In order to be therapeutically effective, the majority of aerosolized antibiotic particles should not have a MMAD larger than 5 μm. When the aerosol contains a large number of particles with a MMAD larger than 5 μm, the larger-sized particles are deposited in the upper airways, decreasing the amount of antibiotic delivered to the site of infection in the lower respiratory tract.

Currently, three types of available nebulizers, jet nebulizers, vibrating porous plate nebulizers and ultrasonic nebulizers, can produce and deliver aerosol particles with diameter sizes between 1 and 5 μm, a particle size that is preferable for treatment of bacterial infections of the lung. Therefore, it would be highly advantageous to provide a macrolide formulation that could be efficiently aerosolized in a jet, vibrating porous plate, and ultrasonic nebulizer. In addition, newer aerosol generating technologies are now available, including mechanical extrusion and both passive and energized dry powder inhalers that are useful for the delivery of therapeutic agents in dry powder form.

Another requirement for an acceptable formulation is adequate shelf life. Generally, antibiotics, and particularly antibiotic solutions for intravenous administration, contain phenol or other preservatives to maintain potency and to minimize the production of degradation products. However, phenol and other preservatives, when aerosolized, may induce bronchospasm, an unwanted occurrence in patients with lung diseases such as chronic bronchitis.

Administration of macrolide antibiotics, such as erythromycylamine, for inhalation in the form of a liquid or dry powder aerosol has the advantage of overcoming poor oral bioavailability associated with the prodrug, while providing efficacious concentrations of the antibiotic to the lung that can not be achieved by either the oral or intravenous route. An additional advantage of aerosol delivery of erythromycylamine is its inherent high affinity for lung tissue and persistence in the plasma compartment (long plasma/tissue half-life). The combination of a high-concentration aerosol delivery, long plasma/tissue half-life and high lung affinity would allow for safer macrolide therapy, which is capable of eradicating or substantially reducing endobronchial infections after a single aerosol dose.

It would be highly advantageous, therefore, to provide macrolide antibiotic formulations, such as erythromycylamine formulations, containing no preservatives, at a pH adjusted to levels that slow or prevent degradation, and are tolerable for a patient, and that provide adequate shelf life suitable for commercial distribution, storage and use.

It is therefore an object of this invention to provide concentrated formulations of macrolide antibiotics, such as erythromycylamine, erythromycin A, roxithromycin, azithromycin and clarithromycin, that contain effective concentrations of the macrolide antibiotic in a form that can be efficiently aerosolized by nebulization, such as by the use of jet, vibrating porous plate, or ultrasonic nebulizers, or dry powder inhalers, into aerosol particle sizes predominantly within a range from 1 and 5 μm.

### SUMMARY OF THE INVENTION

In accordance with the present invention, it has now been discovered that human and non-human animal subjects suffering from or at risk for endobronchial infection, such as an infection by bacterial Streptococcus pneumoniae, Haemophilus influenzae, Staphylococcus aureus, Moraxella catarrhalis and/or the atypical pathogens Legionella pneumonia, Chlamydia pneumoniae, and/or Mycoplasma pneumoniae, can be effectively and efficiently treated by administering to the subject by inhalation an antibacterially effective amount of a macrolide antibiotic, such as erythromycylamine, erythromycin A, roxithromycin, azithromycin or clarithromycin, in a liquid solution or dry powder form suitable for aerosol generation.

Thus, one aspect of the current invention relates to concentrated formulations suitable for efficacious delivery by inhalation of a macrolide antibiotic drug, such as erythromycylamine, erythromycin A, roxithromycin, azithromycin or clarithromycin, into the endobronchial space of a subject suffering from or at risk for a bacterial pulmonary infection.

Another aspect of the invention provides formulations suitable for efficacious delivery of a macrolide antibiotic drug, such as erythromycylamine, erythromycin A, roxithromycin, azithromycin or clarithromycin, into the endobronchial space of a subject suffering from bacterial Streptococcus pneumoniae, Haemophilus influenzae, Staphylococcus aureus, Moraxella catarrhalis and/or the atypical pathogens Legionella pneumonia, Chlamydia pneumoniae, and/or Mycoplasma pneumoniae pulmonary infection.

Another aspect of the current invention provides formulations suitable for efficacious delivery of a macrolide antibiotic drug, such as erythromycylamine, erythromycin A, roxithromycin, azithromycin or clarithromycin, into endobronchial space of a subject to prevent or substantially reduce the risk of pulmonary infection in at-risk patients caused by Stretococcus pneumoniae, Haemophilus influenzae, Staphylococcus aureus, Moraxella catarrhalis and/or the atypical pathogens Legionella pneumonia, Chlamydia pneumoniae, and/or Mycoplasma pneumoniae.

Still another aspect of the current invention provides liquid formulations comprising the equivalent of 50 to 750 mg of a macrolide antibiotic drug, such as erythromycylamine, erythromycin A, roxithromycin, azithromycin or clarithromycin, in 0.5 to 5 ml of a physiologically acceptable carrier, such as saline diluted into a quarter normal saline strength wherein said formulation has a physiologically tolerated osmolarity, salinity, and pH and is suitable for delivery to a subject in concentrated form by aerosol inhalation.

Still another aspect of the current invention provides dry powder formulations comprising the equivalent of 25 to 250 mg of a macrolide antibiotic drug, such as erythromycylamine, erythromycin A, roxithromycin, azithromycin or clarithromycin, in a physiologically acceptable dry powder carrier for delivery to a subject in concentrated form by aerosol inhalation, wherein the dry powder formulations comprise about 50 to 90% by weight of the macrolide antibiotic drug.

Still another aspect of the current invention provides methods for the treatment of pulmonary infections caused by susceptible bacteria by administering to a subject requiring such treatment by inhalation an aerosol formulation comprising an antibacterially effective amount of a macrolide antibiotic drug, such as erythromycylamine, erythromycin A, roxithromycin, azithromycin or clarithromycin, formulated in a physiologically compatible liquid solution or dry powder form, wherein the mass median aerodynamic diameter (MMAD) of particles in the aerosol formulation is predominantly between 1 and 5 μm.

In other aspects, the present invention provides unit dose formulations and devices adapted for use in connection with a high efficiency inhalation system, the unit dose device comprising a container designed to hold and store the relatively small volumes of the macrolide antibiotic formulations of the invention, and to deliver the formulations to an inhalation device for delivery to a subject in aerosol form. In one aspect, a unit dose device of the invention comprises a sealed container, such as an ampoule, containing less than about 2.0 ml of a liquid macrolide antibiotic formulation comprising from about 50 to about 150 mg/ml of a macrolide antibiotic in a physiologically acceptable liquid carrier. Alternatively, the container of the unit dose device may contain less than about 1.5 ml, or less than about 1.0 ml, of the liquid macrolide antibiotic formulation, and the macrolide antibiotic formulation may comprise from about 80 to about 180 mg/ml, or from about 90 to about 120 mg/ml, of macrolide antibiotic. In another aspect, a unit dose device of the invention comprises a sealed container, such as an ampoule, containing a dry powder macrolide antibiotic formulation comprising from about 20 to about 250 mg of a macrolide antibiotic in a physiologically acceptable dry powder carrier. The sealed unit dose containers of the invention are preferably adapted to deliver the macrolide antibiotic formulation to a high efficiency inhalation device for aerosolization and inhalation by a subject.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 illustrates the chemical structure of erythromycylamine and dirithromycin;

FIG. 2 is a graphical representation of the stability of erythromycylamine hydrochloride in aqueous solution at 60, 100, and 150 mg/mL and pH 5.0, 6.0, and 7.0, at 4 degrees centigrade, as described in Example 4;

FIG. 3 is a graphical representation of the stability of erythromycylamine hydrochloride in aqueous solution at 60, 100, and 150 mg/mL and pH 5.0, 6.0, and 7.0, at 25 degrees centigrade, as described in Example 4;

FIG. 4 is a graphical representation of the stability of erythromycylamine hydrochloride in aqueous solution at 60, 100, and 150 mg/mL and pH 5.0, 6.0, and 7.0, at 40 degrees centigrade, as described in Example 4;

FIG. 5 is a graphical representation of the stability of erythromycylamine hydrochloride in aqueous solution at 60, 100, and 150 mg/mL and pH 5.0, 6.0, and 7.0, at 60 degrees centigrade, as described in Example 4;

FIG. 6 is a graphical representation of the stability of erythromycylamine sulfate in aqueous solution at 60, 100, and 150 mg/mL and pH 5.0, 6.0, and 7.0, at 60 degrees centigrade, as described in Example 4;

FIG. 7 is a graphical representation of the stability of erythromycylamine acetate in aqueous solution at 60, 100, and 150 mg/mL and pH 5.0, 6.0, and 7.0, at 60 degrees centigrade, as described in Example 4;

FIG. 8 illustrates mean plasma concentrations of erythromycylamine following a single 25 mg/kg intravenous dose, or single inhalation dose of 30 or 60 mg/ml solution for 30 minutes (0.7 or 1.77 mg/kg pulmonary dose) in rats (n=3), as described in Example 6;

FIG. 9 illustrates mean lung concentrations of erythromycylamine following a single 25 mg/kg intravenous dose, or single inhalation dose of 30 or 60 mg/ml solution for 30 minutes (0.7 or 1.77 mg/kg pulmonary dose) in rats (n=3), as described in Example 6.

FIG. 10 illustrates efficacy of erythromycylamine in the S. pneumoniae pulmonary infection model after 30 minute inhalation administration daily for three (3) days to rats (n=3) and comparing 5 mg/mL (0.13 mg/kg), 25 mg/mL (0.27 mg/kg), and 50 mg/mL (1.3 mg/kg) inhalation dose as described in Example 7; and

FIG. 11 illustrates efficacy of erythromycylamine in the S. pneumoniae pulmonary infection model after 30 minute inhalation administration as a single dose to rats (n=3) and comprising 1 mg/mL (0.03 mg/kg), 5 5 mg/mL (0.13 mg/kg), 25 mg/mL (0.27 mg/kg), and 50 mg/mL (1.3 mg/kg) inhalation dose as described in Example 8.

FIG. 12 illustrates the mean plasma and whole lung concentrations of erythromycylamine following a single dose, 30 minute inhalation administration of a 60 mg/mL sulfate solution in dogs, as described in Example 9.

FIG. 13 illustrates the mean lung concentrations of erythromycylamine in individual lung lobes following a single dose, 30 minute inhalation administration of a 60 mg/mL sulfate solution in dogs as described in Example 9.

### DETAILED DESCRIPTION OF THE PREFERRED EMBODIMENT

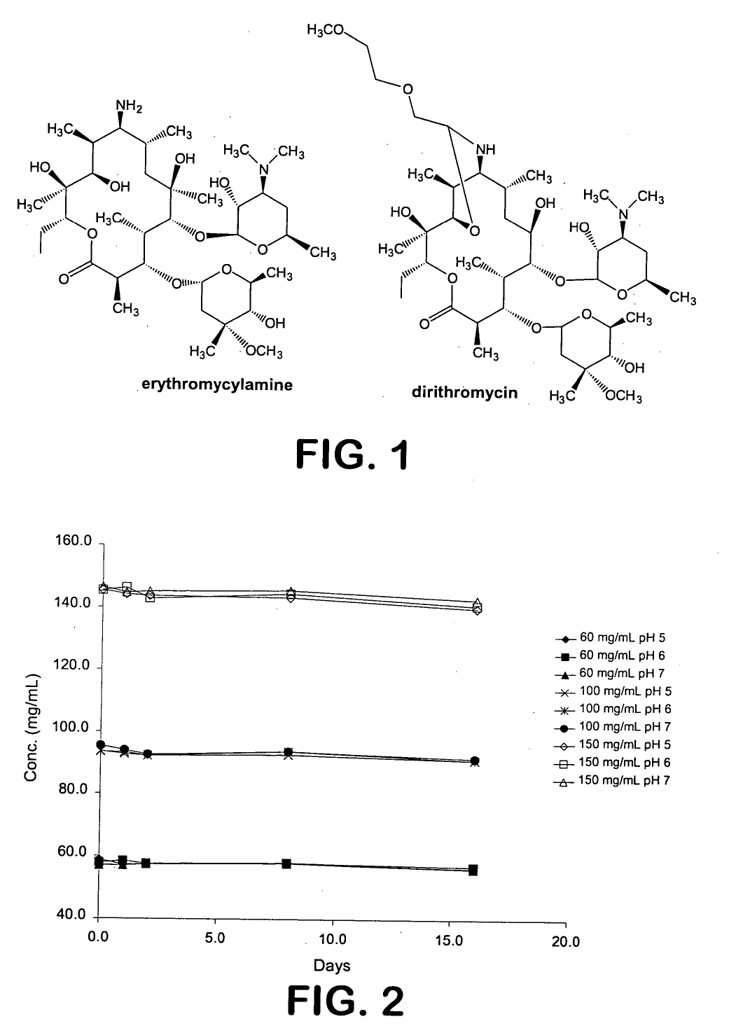

Erythromycylamine and dirithromycin are macrolides having a chemical structure depicted in FIG. 1. Dirithromycin, a prodrug of erythromycylamine, is a broad-spectrum macrolide antibiotic used for treatment of AECB and pneumonia. Macrolide antibiotics useful in the present invention include, for example, erythromycylamine, dirithromycin (a prodrug of erythromycylamine), erythromycin A, clarithromycin (6-O-methyl erythromycin), azithromycin, and roxithromycin. Other newer macrolides such as the ketolides (for example, ABT-773 (39^{th} ICAAC (1999), September 26-29, abstracts F-2133-2141, and HMR-3647 (Drugs of the Future, 23, 591 (1998), 38^{th} ICAAC (1998), September 24-27, abstract A-49), and anhydrolides (see, J. Med. Chem., 1998, 41, 1651-1659 and 1660-1670) may also be used in the practice of the invention. In one aspect of the present invention, the macrolide antibiotic used in the aerosol formulations described herein is erythromycylamine or dirithromycin. Erythromycylamine and dirithromycin have the chemical structures depicted in FIG. 1.

In accordance with the present invention, methods are provided for the treatment of a subject in need of treatment, such as a subject suffering from an endobronchial infection, comprising administering to the subject by inhalation an antibacterially effective amount of a macrolide antibiotic formulation. This aspect of the invention is particularly suitable for formulation of concentrated macrolides, such as erythromycylamine, for aerosolization by small volume, breath actuated, high output rate and high efficiency inhalers to produce a macrolide aerosol particle size between 1 and 5 μm desirable for efficacious delivery of the macrolide into the endobronchial space to treat susceptible microbial infections. The formulations preferably contain minimal yet efficacious amounts of the macrolide formulated in small volumes of a physiologically acceptable solution. For example, an aqueous solution having a salinity adjusted to permit generation of macrolide aerosol particles that are well-tolerated by patients but prevent the development of secondary undesirable side effects such as bronchospasm and cough. By way of example, a quarter normal saline solution is useful for this purpose. By the more efficient administration of the macrolide formulation provided by the present invention, substantially smaller volumes of macrolide than the conventional administration regime are administered in substantially shorter periods of time, thereby reducing the costs of administration and drug waste, and significantly enhancing the likelihood of patient compliance.

Thus, in accordance with one aspect of the present invention, methods are provided for the treatment of a subject in need of treatment, such as a subject suffering from a susceptible endobronchial infection, comprising administering to the subject for inhalation a dose of a nebulized aerosol formulation comprising from about 50 to about 750 mg of a macrolide and a pharmaceutically acceptable carrier. In other aspects of the invention, the aerosol formulations administered in the practice of the invention may be liquid formulations comprising from about 50 to about 150 mg/ml of a macrolide antibiotic, preferably from about 70 to about 130 mg/ml of a macrolide antibiotic, and more preferably from about 90 to about 110 mg/ml of a macrolide antibiotic. Preferably, small volumes of aerosol formulation are administered to the subject. Thus, in this aspect a dose of less than about 2.0 ml of a nebulized liquid aerosol formulation is administered to the subject. In another aspect, a dose of less than about 1.5 ml of a nebulized aerosol formulation is administered to the subject. In yet another aspect, a dose of less than about 1.0 ml of a nebulized aerosol formulation is administered to the subject.

In other aspects, the macrolide compounds of the invention may be formulated for aerosol delivery as a dry powder. As used herein, the term “powder” means a composition that consists of finely dispersed solid particles that are free flowing and capable of being readily dispersed in an inhalation device and subsequently inhaled by a subject so that the particles reach the lungs to permit penetration and deposition in the peripheral airways. Thus, powder formulations of the invention are said to be “respirable.” Preferably the average powder particle size is less than about 10 μm in diameter with a relatively uniform spheroidal shape. More preferably the diameter is less than about 7.5 μm and most preferably less than about 5.0 μm. Usually the particle size distribution is between about 0.1 μm and about 5 μm in diameter, particularly about 1 μm to about 5 μm. Dry powder formulations of the invention have a moisture content such that the particles are readily dispersible in an inhalation device to form an aerosol. This moisture content will generally be below about 10% by weight (% w) water, usually below about 5% w water and preferably less than about 3% w water.

Dry powder formulations of the invention generally comprise a therapeutically effective amount of a macrolide compound of the invention together with a pharmaceutically acceptable carrier. The dry powder formulations of the invention may comprise from about 25 to about 250 mg of a macrolide antibiotic, preferably from about 50 to about 200 mg of a macrolide antibiotic, and more preferably from about 75 to about 150 mg of a macrolide antibiotic. In this aspect of the invention, the dry powder formulations may comprise from about 50% to about 90% by weight of the macrolide antibiotic, preferably from about 60% to about 88% by weight of the macrolide antibiotic, and more preferably from about 75% to about 85% by weight of the macrolide antibiotic.

Suitable pharmaceutically acceptable carriers include carriers that can be taken into the lungs of a patient with no significant adverse toxicological effects on the lungs, including, for example, stabilizers, bulking agents, buffers, salts and the like. A sufficient amount of the pharmaceutically acceptable carrier is employed to obtain desired stability, dispersibility, consistency and bulking characteristics to ensure a uniform pulmonary delivery of the composition to a subject in need thereof. The actual amount of pharmaceutically acceptable carrier employed may be from about 0.05% w to about 99.95% w. More preferably, from about 5% w to about 95% w of the pharmaceutically acceptable carrier will be used. Most preferably, from about 10% w to about 90% w of the pharmaceutically acceptable carrier will be used.

Pharmaceutical excipients useful as carriers in this invention include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two. Preferred bulking agents include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-β-cyclodextrin; and polysaccharides, such as raffinose, maltodextrins, dextrans, and the like; alditols, such as mannitol, xylitol, and the like. A preferred group of carbohydrates includes lactose, threhalose, raffinose maltodextrins, and mannitol. Suitable polypeptides include aspartame. Amino acids include alanine and glycine, with glycine being preferred. Additives, which may be included as minor components of the dry powder formulations of the invention, may be included for conformational stability during spray drying and for improving dispersibility of the powder. These additives include hydrophobic amino acids such tryptophan, tyrosine, leucine, phenylalanine, and the like. Suitable pH adjusters or buffers include organic salts prepared from organic acids and bases, such as sodium citrate, sodium ascorbate, and the like; sodium citrate is preferred.

In other aspects, the present inventions relates to concentrated macrolide formulations, such as a concentrated erythromycylamine formulation, suitable for efficacious delivery of the macrolide by aerosolization into endobronchial space. The invention is suitable for formulation of concentrated erythromycylamine for aerosolization by jet, vibrating porous plate, ultrasonic or dry powder nebulizers to produce erythromycylamine aerosol particle size between 1 and 5 μm preferable for efficacious delivery of erythromycylamine into the endobronchial space to treat Streptococcus pneumoniae, Haemophilus influenzae, Staphylococcus aureus Moraxella catarrhalis and Legionella pneumonia, Chlamydia pneumoniae, and Mycoplasma pneumoniae infections. The formulations preferably contain minimal, yet efficacious amounts of erythromycylamine formulated in a relatively small volume of physiologically acceptable solution having a salinity, or a dry powder, adjusted to permit generation of an erythromycylamine aerosol that is well-tolerated by patients but preventing the development of secondary undesirable side effects such as bronchospasm and cough.

Primary requirements for any aerosolized formulation are its safety and efficacy. Additional advantages are lower treatment cost, practicality of use, long-shelf life, storage and optimization of nebulizer.

The aerosol formulation is nebulized predominantly into particle sizes which can be delivered to the terminal and respiratory bronchioles where the Streptococcus pneumoniae, Haemophilus influenzae, Staphylococcus aureus, and Moraxella catarrhalis and the atypical bacteria Legionella pneumonia, Chlamydia pneumoniae, and Mycoplasma pneumoniae or other susceptible bacteria reside in patients with chronic bronchitis and pneumonia. Streptococcus pneumoniae, Haemophilus influenzae, Staphylococcus aureus, Moraxella catarrhalis, Legionella pneumonia, Chlamydia pneumoniae, and Mycoplasma pneumoniae are present throughout the airways including the bronchi, bronchioli and lung parenchema. However, they are most predominant in terminal and respiratory bronchioles. During exacerbation of infection, bacteria can also be present in alveoli. Therefore, in one aspect, the present invention provides a formulation that is delivered throughout the endobronchial tree to the terminal bronchioles and eventually to the parenchymal tissue.

Aerosolized erythromycylamine formulation is formulated for efficacious delivery of erythromycylamine to the lung endobronchial space. A specific jet, vibrating porous plate or ultrasonic nebulizer is selected to allow the formation of an erythromycylamine aerosol particles with a mass median aerodynamic diameter predominantly between 1 to 5 μm. The formulated and delivered amount of erythromycylamine is efficacious for treatment and/or prophylaxis of endobronchial infections, particularly those caused by the bacteria Streptococcus pneumoniae, Haemophilus influenzae, Staphylococcus aureus, and Moraxella catarrhalis and the atypical pneumonias Legionella pneumonia, Chlamydia pneumoniae, and Mycoplasma pneumoniae. The formulation has salinity adjusted to permit generation of erythromycylamine aerosol well tolerated by patients. Further, the formulation has suitable osmolarity. The formulation has a small aerosolizable volume and is able to deliver an effective dose of erythromycylamine to the site of the infection. Additionally, the aerosolized formulation does not impair negatively the function of the airways by causing undesirable side effects.

The antibiotic formulation may be administered with the use of an inhalation device having a relatively high rate of aerosol output, high emitted dose efficiency, and emission limited to periods of actual inhalation by the patient. Thus, while conventional air-jet nebulizers exhibit a rate of aerosol output on the order of 3 μl/sec, inhalation devices useful for use in the practice of the present invention will typically exhibit a rate of aerosol output of not less that about 5 μl/sec, more preferably not less than about 6.5 μl/sec, and most preferably not less than about 8 μl/sec. In addition, while conventional air-jet nebulizers have a relatively low emitted dose efficiency and typically release about 55% (or less) of the nominal dose as aerosol, inhalation devices useful for use in the practice of the present invention will typically release at least about 75%, more preferably at least about 80% and most preferably at least about 85% of the loaded dose as aerosol for inhalation by the subject. In other aspects, conventional air-jet nebulizers typically continually release aerosolized drug throughout the delivery period, without regard to whether the subject is inhaling, exhaling or in a static portion of the breathing cycle, thereby wasting a substantial portion of the loaded drug dose. In contrast, preferred inhalation devices for use in the present invention will be breath actuated, and restricted to delivery of aerosolized particles of the macrolide formulation to the period of actual inhalation by the subject. A representative inhalation device meeting the above criteria and suitable for use in the practice of the invention is the Aerodose™ inhaler, available from Aerogen, Inc., Sunnyvale, Calif. The Aerodose™ inhaler generates an aerosol using a porous membrane driven by a piezoelectric oscillator. Aerosol delivery is breath actuated, and restricted to the inhalation phase of the breath cycle, i.e., aerosolization does not occur during the exhalation phase of the breath cycle. The airflow path design allows normal inhale-exhale breathing, compared to breath-hold inhalers. Additionally, the Aerodose™ inhaler is a hand-held, self-contained, and easily transported inhaler. Although piezoelectric oscillator aerosol generators, such as the Aerodose™ inhaler, are presently preferred for use in the practice of the invention, other inhaler or nebulizer devices may be employed that meet the above performance criteria and are capable of delivering the small dosage volumes of the invention with a relative high effective deposition rate in a comparatively short period of time.

In other aspects of the present invention, unit dose formulations and devices are provided for administration of a macrolide antibiotic formulation to a subject with an inhaler, in accordance with the methods of the invention as described supra. Preferred unit dose devices comprise a container designed to hold and store the relatively small volumes of the macrolide antibiotic formulations of the invention, and to deliver the formulations to an inhalation device for delivery to a patient in aerosol form. In one aspect, unit dose containers of the invention comprise a plastic ampoule filled with a macrolide antibiotic formulation of the invention, and sealed under sterile conditions. Preferably, the unit dose ampoule is provided with a twist-off tab or other easy opening device for opening of the ampoule and delivery of the macrolide antibiotic formulation to the inhalation device. Ampoules for containing drug formulations are well known to those skilled in the art (see, for example, U.S. Pat. Nos. 5,409,125, 5,379,898, 5,213,860, 5,046,627, 4,995,519, 4,979,630, 4,951,822, 4,502,616 and 3,993,223, the disclosures of which are incorporated herein by this reference). The unit dose containers of the invention may be designed to be inserted directly into an inhalation device of the invention for delivery of the contained macrolide antibiotic formulation to the inhalation device and ultimately to the subject.

In accordance with this aspect of the invention, a unit dose device is provided comprising a sealed container containing less than about 5.0 ml, preferably less than about 3.0 ml and most preferably less than about 2.0 ml of a liquid macrolide antibiotic formulation comprising from about 50 to about 150 mg/ml of a macrolide antibiotic in a physiologically acceptable carrier, the sealed container being adapted to deliver the macrolide antibiotic formulation to an inhalation device for aerosolization. Suitable macrolide antibiotics for use in connection with this aspect of the invention include those macrolide antibiotics described in detail, supra. In a presently preferred embodiment, the macrolide antibiotic employed in the unit dose devices of the invention is erythromycylamine. In other aspects of the invention, the unit dose devices of the invention may contain a liquid macrolide antibiotic formulation comprising from about 70 to about 130 mg/ml of macrolide antibiotic. In yet other aspects of the invention, the unit dose devices of the invention may contain a liquid macrolide antibiotic formulation comprising from about 90 to about 110 mg/ml of macrolide antibiotic.

In preferred liquid unit dose formulations of the invention, the physiologically acceptable carrier may comprise a physiological saline solution such as a solution of one quarter strength of normal saline, having a salinity adjusted to permit generation of erythromycylamine aerosol well-tolerated by patients but to prevent substantially the development of secondary undesirable side effects such as bronchospasm and cough.

In yet other aspects of the invention, dry powder formulations of the invention are placed within a suitable unit dose receptacle in an amount sufficient to provide a subject with a macrolide antibiotic compound of the invention for a unit dosage treatment by dry powder inhalation. Preferred dry powder unit dosage receptacles fit within a suitable inhalation device to allow for the aerosolization of the macrolide-based dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the formulations known in the art such as gelatin or plastic capsules with a removable portion that allows a stream of gas (e.g., air) to be directed into the container to disperse the dry powder formulation. Such containers are exemplified by those shown in U.S. Pat. Nos. 4,227,522, 4,192,309, and 4,105,027. Suitable containers also include those used in conjunction with Glaxo's Ventolin Rotohaler brand powder inhaler or Fison's Spinhaler brand powder inhaler. Another suitable unit-dose container which provides a superior moisture barrier is formed from an aluminum foil plastic laminate. The macrolide powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler.RTM. (U.S. Pat. Nos. 4,627,432, 4,811,731; and 5,035,237). All of these references are incorporated herein by reference.

In accordance with this aspect of the invention, a unit dose device is provided comprising a sealed container containing a dry powder formulation comprising from about 25 to about 250 mg of a macrolide antibiotic, preferably from about 50 to about 200 mg of a macrolide antibiotic, and more preferably from about 75 to about 150 mg of a macrolide antibiotic in a physiologically acceptable dry powder carrier, the sealed container being adapted to deliver the macrolide antibiotic formulation to an inhalation device for aerosolization. In this aspect of the invention, the dry powder formulations may comprise from about 50% to about 90% by weight of the macrolide antibiotic, preferably from about 60% to about 88% by weight of the macrolide antibiotic, and more preferably from about 75% to about 85% by weight of the macrolide antibiotic.

Aerosol Erythromycylamine Formulation

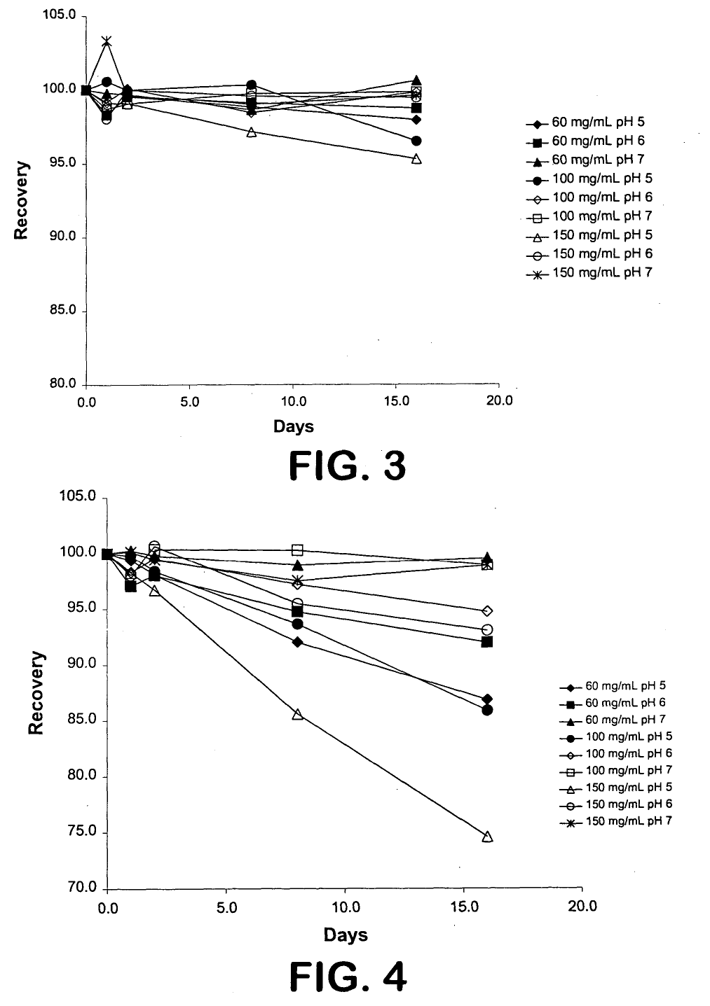

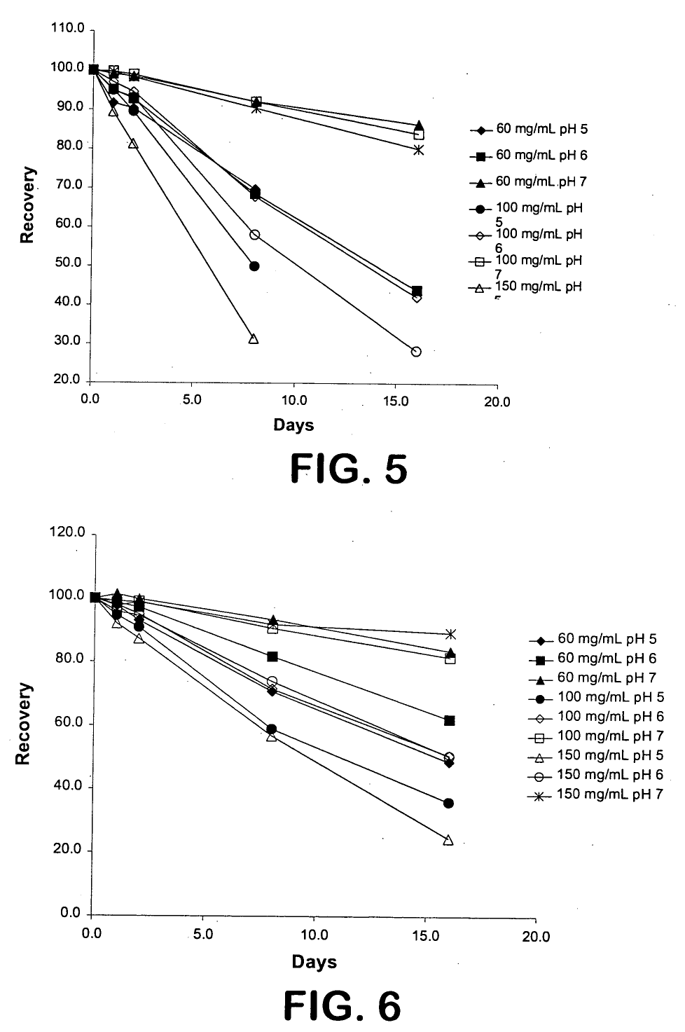

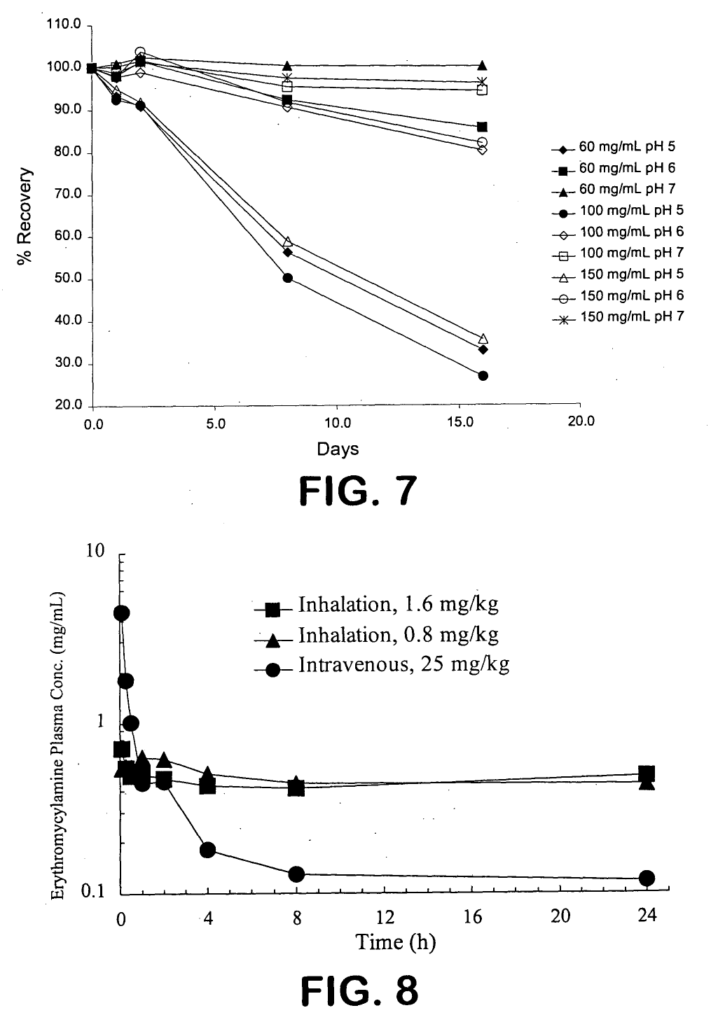

In order to assess the stability of erythromycylamine in aqueous solutions three salt forms of the antibiotic were prepared and submitted to varying conditions of temperature, time, concentration, and pH. Erythromycylamine concentrations were determined by HPLC methodology. The data from these stability studies are shown in FIGS. 2-7 and several important findings are revealed. First, the stability of erythromycylamine hydrochloride as expected, was directly proportional to temperature of the solution (see FIGS. 2-5). Second, erythromycylamine solutions were more stable at neutral pH 7 than acidic pH 5 and 6 (FIG. 5). This result is consistent with the known effects of pH on the degradation of macrolide antibiotics. One of the main degradation pathways is loss of the neutral sugar, cladinose (see J. Chrom. A, 812m 1998, 255-286). Third, solutions of erythromycylamine acetate were more stable at pH 6 and 7 than the corresponding hydrochloride and sulfate salts at the same pH (compare FIG. 7 to FIGS. 5 and 6).

Liquid and dry powder formulations according to the invention contain from about 50 to about 750 mg, preferably from about 75 to about 600 mg, and most preferably from about 100 to about 500 mg of a macrolide antibiotic drug, such as erythromycylamine acetate, per dose. This corresponds to minimal yet efficacious amounts of erythromycylamine to suppress Streptococcus pneumoniae, Haemophilus influenzae, Staphylococcus aureus, Moraxella catarrhalis, Legionella pneumonia, Chlamydia pneumoniae, and Mycoplasma pneumoniae infections in the endobronchial space.

Presently preferred liquid aerosol erythromycylamine formulations according to the invention comprise from about 90 to about 110 mg of erythromycylamine sulfate per 1 mL of quarter normal saline. This corresponds to a representative efficacious amount of erythromycylamine to suppress bacterial infections of AECB.

Both patients and aerosol generating devices are sensitive to the osmolarity, pH, and ionic strength of the formulation. It has now been discovered that this problem is conveniently solved by formulating erythromycylamine solutions in quarter normal saline, that is saline containing 0.225% of sodium chloride, and that quarter normal saline is a suitable vehicle for delivery of erythromycylamine into the endobronchial space.

Chronic bronchetic patients and other patients with chronic endobronchial infections have a high incidence of bronchospastic or asthmatic airways. These airways are sensitive to hypotonic and hypertonic aerosols, to the concentration of a permeant ion, particularly a halide such as chloride, as well as to aerosols that are acidic or basic. The effects of irritating the airways can be clinically manifested by cough or bronchospasm. Both of these conditions can prevent efficient delivery of aerosolized erythromycylamine into the endobronchial space.

The erythromycylamine acetate, hydrochloride, and sulfate formulation containing 60-100 mg of erythromycylamine per ml of quarter normal saline has an osmolarity in the range of 130-400 mOsm/kg. This is within the safe range of aerosols administered to a chronic bronchitis patient (Table 1).

TABLE 1
Osmolality of Erythromycylamine Solutions as
a Function of Salt Form, pH, and Concentration:
Experimental and Theoretical Results

| | | Conc. | Experimental | Theoretical |
|---|---|---|---|---|
| Salt | pH | (mg/ml) | (mOsm/kg) | (mOsm/kg) |
| Acetate | 5.0 | 60 | 300 | 245 |
| | | 100 | 518 | 408 |
| | | 150 | 840 | 613 |
| Acetate | 6.0 | 60 | 365 | |
| | | 100 | 639 | |
| | | 150 | 701 | |
| Acetate | 7.0 | 60 | 501 | |
| | | 100 | 891 | |
| | | 150 | 746 | |
| Hydrochloride | 5.0 | 60 | 227 | 245 |
| | | 100 | 382 | 408 |
| | | 150 | 601 | 613 |
| Hydrochloride | 6.0 | 60 | 224 | |
| | | 100 | 382 | |
| | | 150 | 598 | |
| Hydrochloride | 7.0 | 60 | 226 | |
| | | 100 | 386 | |
| | | 150 | 594 | |
| Sulfate | 5.0 | 60 | 130 | 163 |
| | | 100 | 239 | 272 |
| | | 150 | 396 | 409 |
| Sulfate | 6.0 | 60 | 132 | |
| | | 100 | 238 | |
| | | 150 | 395 | |
| Sulfate | 7.0 | 60 | 132 | |
| | | 100 | 241 | |
| | | 150 | 391 | |

The pH of the formulation is equally important for aerosol delivery. As noted previously, when the aerosol is either acidic or basic, it can cause bronchospasm and cough. The safe range of pH is relative; some patients will tolerate a mildly acidic aerosol that in others will cause bronchospasm. Any aerosol with a pH of less than 4.5 usually will induce bronchospasm in a susceptible individual; aerosols with a pH between 4.5 and 5.0 will occasionally cause this problem. An aerosol with a pH between 5.0 and 7.0 is considered to be safe. Any aerosol having pH greater than 10.0 is to be avoided since irritation resulting in bronchospasm may occur. The optimum pH for the aerosol formulation was determined to be between pH 5.0 and 7.0.

In one aspect, liquid formulations of the invention are preferably nebulized predominantly into particle sizes allowing a delivery of the drug into the terminal and respiratory bronchioles and lower airways where the bacteria reside. For efficacious delivery of erythromycylamine to the lung endobronchial airway space by aerosol, the formation of aerosol particles having a mass median aerodynamic diameter predominantly between 1 to 5 μm is necessary. The formulated and delivered amount of erythromycylamine for treatment and prophylaxis of endobronchial infections, particularly those caused by the bacteria Streptococcus pneumoniae, Haemophilus influenzae, Staphylococcus aureus, Moraxella catarrhalis, Legionella pneumonia, Chlamydia pneumoniae, and Mycoplasma pneumoniae, must effectively target the endobronchial surface. Delivered doses of the formulations preferably have the smallest practical aerosolizable volume able to deliver an effective dose of erythromycylamine to the site of the infection. Preferred formulations additionally provide conditions that do not adversely affect the functionality of the airways. Consequently, preferred formulations contain a sufficient amount of the drug formulated under conditions that allow its efficacious delivery, while avoiding undesirable reactions. The new formulations according to the invention meet all these requirements.

According to the invention, erythromycylamine is formulated in a dosage form intended for inhalation therapy by patients with chronic bronchitis and pneumonia. Since the patients reside throughout the world, it is desirable that the formulation has reasonably long shelf life. Storage conditions and formulation stability thus become important.

As discussed above, the pH of the solution is important. A pH between 5.0 and 7.0, preferably about 6.0, is optimal from the storage and longer shelf-life point of view.

The formulation is typically stored in a one- to two-milliliter low-density polyethylene (LDPE) vials. The vials are aseptically filled using a blow-fill-seal process. The vials are sealed in foil overpouches.

Stability of the formulation with respect to oxidation is another very important issue. If the drug is degraded before aerosolization, a smaller amount of the drug is delivered to the lung, thus impairing the treatment as well as provoking conditions that could lead to the development of resistance to erythromycylamine, because the delivered dose would be too small. Moreover, erythromycylamine degradation products may provoke bronchospasm and cough. To prevent oxidative degradation of erythromycylamine and in order to provide acceptable stability, a product with low oxygen content is produced by packaging the LDPE vials in oxygen-protective packaging comprising foil overpouches, six vials per overpouch. Prior to vial filling, the solution in the mixing tank is nitrogen sparged and the annular overpouch headspace is nitrogen purged. In this way, both hydrolysis and oxidation of erythromycylamine is prevented.

II. Aerosolization Devices

Aerosolization devices, such as a jet, vibrating porous plate or ultrasonic nebulizers, useful in the practice of the invention are generally able to nebuliize the formulation of the invention into aerosol particles predominantly in the range from 1-5 μm. Predominantly in this application means that at least 70% but preferably more than 90% of all generated aerosol particles are within 1-5 μm range.

Nebulizers such as jet, ultrasonic, vibrating porous plate, and energized dry powder inhalers, that can produce and deliver particles between the 1 and 5 μm particle size that is optimal for treatment of Streptococcus pneumoniae, Haemophilus influenzae, Staphylococcus aureus, Moraxella catarrhalis Legionella pneumonia, Chlamydia pneumoniae, and Mycoplasma pneumoniae infections, are currently available or can be produced using known methods and materials. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. However, only some formulations of erythromycylamine can be efficiently nebulized by these three nebulizers, as these devices are sensitive to the pH and ionic strength of the formulation.

While a variety of devices are available, only a limited number of these nebulizers are suitable for the purposes of this invention. Preferred nebulizers useful in the present invention include, for example, AeroNeb™ and AeroDose™ vibrating porous plate nebulizers (AeroGen, Inc., Sunnyvale, Calif.), Sidestream® nebulizers (Medic-Aid Ltd., West Sussex, England), Pari LC Plus® and Pari LC Star® jet nebulizers (Pari Respiratory Equipment, Inc., Richmond, Va.), and Aerosonic™ (DeVilbiss Medizinische Produkte (Deutschland) GmbH, Heiden, Germany) and UltraAire® (Omron Healthcare, Inc., Vernon Hills, Ill.) ultrasonic nebulizers.

III. Aerosol Pharmacokinetics

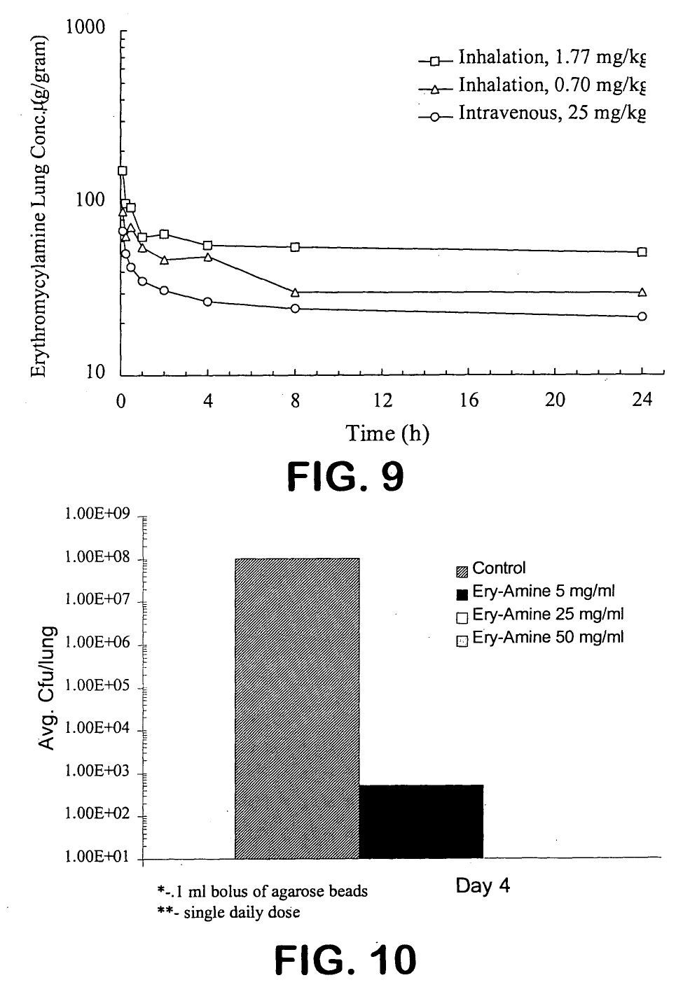

Solutions of erythromycylamine were administered to rats by the IV and inhalation routes and drug concentrations in plasma and lung were measured. Data from these studies is shown in FIGS. 8 and 9. Two dose levels were selected for the inhalation delivery route, 1.7 and 0.7 mg/kg, and were compared to a single intravenous dose (25 mg/kg).

Dose normalized AUC of erythromycylamine in the lung for IV (25 mg/kg), inhalation (1.7 mg/kg), and (0.7 mg/kg) was 24.21, 1067.84 and 848.34 μg·h/gram, respectively. Therefore, by administering erythromycylamine directly to the lung via inhalation route, lung drug levels achieved were approximately 40 times higher on a milligram basis than by the intravenous route of delivery. Thus, antibiotic therapy by inhalation should be more efficacious than treatment by oral or IV routes.

IV. Aerosol Efficacy

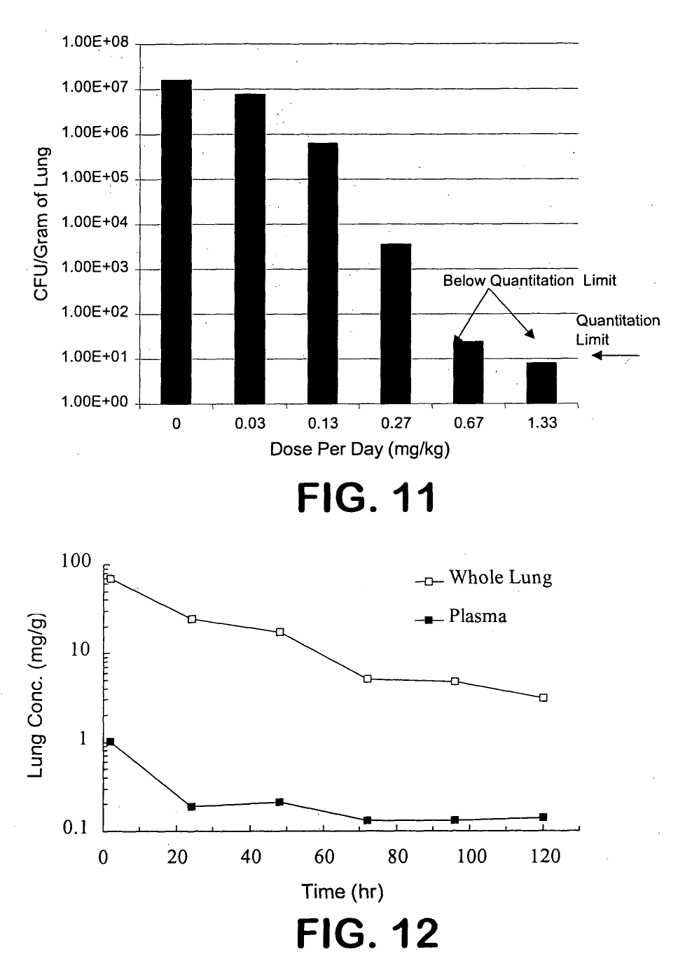

Erythromycylamine was very effective by both intravenous and aerosol administration. At the lowest dose tested (10 mg/kg per day) intravenous erythromycylamine reduced the lung burden of S. pneumonia to below the limits of detection (10 CFU/gram of lung) as shown in Example 7. Aerosol was also very effective (see FIG. 10) with only detectable recovery of S. pneumonia at 5 mg/ml aerosol solution (calculated dose 0.13 mg/kg per day). In addition, erythromyclamine was very effective when administered as a single aerosol dose at concentrations greater than those required for single daily doses for 3 days. A single dose of 0.13 mg/kg was less effective (less than 2 orders of magnitude reduction in CFU/gram) compared with 0.13 mg/kg for three consecutive days (5 orders of magnitude reduction). However, a single dose of 0.67 mg/kg achieved almost complete clearance of the organism from lung tissue, an effect similar to the multiple dose efficacy indicating that at this concentration, the second and third doses added little value (see FIG. 11).

The pharmacokinetic evaluation of aerosolized erythromycylamine suggests, and the efficacy data indicates, that equivalent lung concentrations to multiple daily IV, oral, or aerosol doses can be achieved by a single aerosol dose and that the single dose would be about 3-5 fold greater than required for similar effectiveness as three daily doses.

Utility

One aspect of the utility of this invention is that small volume, high concentration formulations of macrolide antibiotics, such as erythromycylamine, can be used with suitable nebulizers to deliver an efficacious dose of erythromycylamine to the endobronchial space in people with chronic bronchitis, bronchiectasis, and pneumonia caused by macrolide susceptible bacteria or other infections. The formulation is safe and very cost effective. Furthermore, the formulations may be kept in a nitrogen environment, with pH controlled for tolerance, to provide adequate shelf life for commercial distribution.

### EXAMPLE 1

### General Procedure for the Preparation of Erythromycylamine Salts Synthesis of Erythromycyclamine Acetate

To a solution of 10.0 g (13.6 mmol) of erythromyclamine in 100 mL of MeOH cooled in an ice bath was added dropwise 1.56 mL (27.2 mmol, 2.0 eq) of glacial acetic acid. The solution was warmed to ambient over a period of 30 min, then the solvent removed under reduced pressure. Et₂O (50 mL) was added and the slurry concentrated. This was repeated to provide 11.52 g (96.9%) of erythromyclamine acetate monohydrate as a white powder; IR (KBr, cm⁻¹) 1718, 1560, 1406, 1168, 1080, 1055, 1012; ¹H NMR (400 MHz, CD₃OD) δ0.89 (t, 3H, J=7.2 Hz), 1.06-1.32 (m, 27H), 1.35-1.47 (m, 4H), 1.52-1.66 (m, 3H), 1.85-2.02 (m, 8H), 2.03-2.26 (m, 2H), 2.45-2.49 (m, 1H), 2.66-2.77 (m, 5H), 2.91-3.09 (m, 3H), 3.21-3.40 (m, 6H), 3.58 (d, 1H, J=7.0 Hz), 3.67 (s, 1H), 3.78-3.83 (m, 2H), 4.10-4.13 (m, 1H), 4.59 (d, 1H, J=7.0 Hz), 4.88-5.01 (m, 12H); MS m/z 735.6 (M⁺-2AcOH-2H₂O); KF 2.33% H₂O.

Anal. Calcd for C₄₁H₈₀N₂O₁₇: C, 56.40; H, 9.24; N, 3.21. Found: C, 56.38; H, 9.21; N, 3.16.

### EXAMPLE 2

### Synthesis of Erythromycylamine Sulfate

To a solution of 10.0 g (13.6 mmol) of erythromyclamine in 100 mL of MeOH cooled in an ice bath was added dropwise 0.73 mL (13.6 mmol, 1.0 eq) of concentrated sulfuric acid. The solution was warmed to ambient over a period of 30 min, then the solvent removed under reduced pressure. Et₂O (50 mL) was added and the slurry concentrated. This was repeated to provide 11.13 g (96.1%) of erythromyclamine sulfate monohydrate as a white powder; IR (KBr, cm⁻¹) 1718, 1384, 1168, 1122, 1078, 1012; ¹H NMR (400 MHz, CD₃OD) δ0.89 (t, 3H, J=7.2 Hz), 1.08-1.32 (m, 27H), 1.45-1.63 (m, 7H), 1.89-2.04 (m, 2H), 2.23-2.31 (m, 2H), 2.44-2.47 (m, 1H), 2.84-2.89 (5H), 2.99-3.07 (m, 3H), 3.30-3.49 (m, 6H), 3.58 (d, 1H, J=7.0 Hz), 3.69 (s, 1H), 3.78-3.86 (m, 2H), 4.09-4.11 (m, 1H), 4.60 (d, 1H, J=6.8 Hz), 4.87-4.99 (m, 12H); MS m/z 735.7 (M⁺-H₂SO₄-2H₂O); KF 2.93 % H₂O.

Anal. Calcd for C₃₇H₇₄N₂O₁₆S: C, 52.22; H, 8.76; N, 3.29. Found: C, 52.55; H, 8.91; N, 3.27.

### EXAMPLE 3

### Synthesis of Erythromycylamine Hydrochloride

To a solution of 10.0 g (13.6 mmol) of erythromyclamine in 100 mL of MeOH cooled in an ice bath was added dropwise 2.34 mL (27.2 mmol, 2.0 eq) of 37% hydrochloric acid. The solution was warmed to ambient over a period of 30 min, then the solvent removed under reduced pressure. Et₂O (50 mL) was added and the slurry concentrated. This was repeated to provide 11.24 g (97.9%) of erythromyclamine hydrochloride dihydrate as a white powder; IR (KBr, cm⁻¹) 1718, 1466, 1383, 1170, 1078, 1055, 1011; ¹H NMR (400 MHz, CD₃OD) δ0.87-0.91 (m, 3H), 1.10-1.31 (m, 27H), 1.43-1.65 (m, 7H), 1.89-2.01 (m, 2H), 2.25-2.27 (m, 2H), 2.45-2.48 (m, 1H), 2.82-3.10 (m, 8H), 3.34-3.42 (m, 8H), 3.57-3.58 (m, 1H), 3.67 (s, 1H), 3.80-3.82 (m, 2H), 4.08-4.11 (m, 1H), 4.61-5.00 (m, 13H); MS m/z 735.6 (M⁺-2HCl-2H₂O); KF 4.38% H₂O.

Anal. Calcd for C₃₇H₇₆Cl₂N₂O₁₂: C, 52.65; H, 9.08; N, 3.32. Found: C, 52.21; H, 9.18; N, 3.20.

### EXAMPLE 4

### Aqueous Formulation and Stability of Erythromycylamine Salts

Preparation of Solutions Erythromyclamine (9.0 g, 12.2 mM) free base was added to a tared 100 mL Erlenmeyer flask. De-ionized water (25 mL) was added to the flask with agitation by magnetic stirrer. 1N sulfuric acid (24.5 mL, 2 equivalents) was gradually added while stirring. When the solution was clear, it was removed from the stir plate and re-weighed. Deionized water was added dropwise to obtain a final solution weight of 62.9 g. The solution was divided into three 20 mL portions, and the pH was adjusted to the desired value (5.0, 6.0, or 7.0) by dropwise addition of 1N sodium hydroxide or sulfuric acid while monitoring with a pH meter. The above procedure was used to prepare solutions at 100 mg/mL and 60 mg/mL by adjusting the weight of erythromyclamine (6.0 g and 3.6 g) and the volume of 1N sulfuric acid (16.3 and 9.8 mL).

Solutions of the acetate and hydrochloride salts of erythromyclamine at 150, 100 and 60 mg/mL were prepared as described above, except that 1N Acetic acid and 1N Hydrochloric acid (2 equivalents) were added to prepare the salts and adjust the pH.

Aliquots of each salt form at each concentration and each pH were stored at 4, 40, and 60° C., and at ambient temperature.

Stability Determination All solutions were analyzed immediately after preparation (t=0) and at 24 hours, 48 hours, eight days, 15 days and 22 days following preparation, excepting that samples that appeared substantially degraded at eight days were omitted from subsequent analyses.

Refrigerated and heated samples were equilibrated to ambient temperature for at least one hour prior to sample preparation. Final dilution volume for all samples was 10 mL. The diluent for all samples consists of an 80:20 (v/v) mixture of 50 mM phosphate buffer at pH 6.5 and acetonitrile.

An appropriate amount of sample (40 microliters for a 150 mg/mL solution, 50 microliters for a 100 mg/mL, or 100 microliters for a 60 mg/mL solution) was transferred to a 20 mL scintillation vial. 10 mL of the diluent were added to the vial and mixed thoroughly.

Standard Preparation Standards were prepared in duplicate and used for a maximum of three days. Ery-amine free base (30 mg) was transferred to a tared 50 mL volumetric flask and exact weight was recorded. Sample diluent (45 mL) is added and sonicated briefly to dissolve. The standard was cooled and diluted to volume with diluent.

Sample and Standard Analysis Samples and standards were analyzed by reversed-phase high performance liquid chromatography. A 250×4.6 mm Phenomenex Luna CN column with 5 micron particle size was used to perform the separation. All analyses were performed on an Agilent Technologies HP1100 chromatography system, and the data were acquired and stored using an Agilent Technologies ChemStation data system. Analytical parameters were as shown below in Table 2.

TABLE 2

| | |
|---|---|
| Flow Rate | 1.0 mL/min |
| Column Temperature | 30° C. |
| Injection Volume | 20 μL |
| Detector | UV absorbance at 200 nm |
| Run Time | 10 min |
| Mobile Phase A | 50 mM Phosphate pH 2.1 |
| Mobile Phase B | Acetonitrile |
| Composition | 80/20 A/B |

### EXAMPLE 5

### Osmolality of Erythromyclamine Salt Solutions

Three portions of erythromyclamine HCl salt (0.6 g, 1.0 g and 1.5 g) were weighed into separate 10 mL volumetric flasks. Easypure UV water (8 mL) was added to each flask and sonicated until completely dissolved, then diluted to volume. This procedure was repeated for the Erythromyclamine sulfate and acetate. The pH and osmolality of each solution were measured, and the measured osmolality compared to the theoretical values.

The salts prepared in Example 4 (4° C.) were allowed to equilibrate to room temperature, and the osmolality was measured. The results are shown in Table 3:

TABLE 3
Erythromyclamine Salt Osmolality Study

| | | | | | |
|---|---|---|---|---|---|
| | | Theoretical | Actual | | |
| Target | Actual | Osmolarity | Osmolarity | | |
| Weight (g) | Weight (g) | (mOsm) | (mOsm) | pH | Comments |

Erythromyclamine HCl Salt Lot # TEM-702-171
dihydrate mw = 843.91 g/m

| | | | | | |
|---|---|---|---|---|---|
| 0.600 | 0.59957 | 279 | 213 | 7.57 | wh. cloudy |
| 1.00 | 1.00443 | 474 | 357 | 7.59 | wh. cloudy |
| 1.50 | 1.50258 | 733 | 534 | 7.60 | wh. cloudy |

Erythromyclamine Sulfate Salt Lot # TEM-702-169
monohydrate mw = 883.14 g/m

| | | | | | |
|---|---|---|---|---|---|
| 0.600 | 0.60368 | 146 | 137 | 77.62 | wh. cloudy |
| 1.00 | 1.00430 | 258 | 227 | 77.63 | wh. cloudy |
| 1.50 | 1.49941 | 408 | 340 | 77.61 | wh. cloudy |

Erythromyclamine Acetate Salt Lot # TEM-702-167
monohydrate mw = 873.08 g/m

| | | | | | |
|---|---|---|---|---|---|
| 0.600 | 0.60189 g | 195 | 207 | 6.62 | sl. cloudy |
| 1.00 | 1.00713 g | 345 | 346 | 6.67 | sl. cloudy |
| 1.50 | 1.50178 g | 552 | 516 | 6.62 | si. cloudy |

(((wt. in grams/molecular wt )*number of species)/0.01 L)*(1000 mOsm/1 Osm)= X mOsm

### EXAMPLE 6

### Aerosol Delivery of Erythromycylamine to Rats Characterization of Aerosol Pharmacokinetics

IV Pharmacokinetics: Erythromycylamine (250 mg) was dissolved in a 5 mL of DI water, and 12 mL of concentrated sulfuric acid was added. A solution of dilute sulfuric acid (1:10 v/v) was added gradually to the solution to dissolve the drug completely. The solution of dilute sulfuric acid was added gradually to bring the pH of solution to 6.8-7.2. By adding DI water, the total volume of solution was brought up to 8 mL. A 200 μL solution of erythromycylamine sulfate (25 mg/kg) was delivered to male Sprague-Dawley rats (Simonsen Laboratories, 1180 C Day Road, Gilroy, Calif. 95020) by intravenous administration via the lateral tail vein. Animals were anesthetized with 1-4% isoflurane and lung and blood samples were collected from 3 rats at 0.083, 0.25, 0.5 1, 2, 4, 6, 8 and 24 hours post dosing. The blood samples were collected via cardiac puncture using heparin as an anticoagulant. Lungs were removed surgically following blood sampling, and the bronchi and trachea were removed and discarded. The remaining lung tissue was processed as described below. Both the lung and blood samples were immediately placed on ice, and the blood samples were centrifuged immediately following collection to harvest plasma samples. Both lung and plasma samples were stored at −80° C. until assayed.

Erythromycylamine concentrations in plasma and the lung (per gram of lung tissue) were determined using a validated LC-MS method. Plasma samples (100 μg) were spiked with oleandomycin (internal standard, 1 μg/mL) before extraction. Plasma samples (100 μL) were deproteinated with 3.3% trichloroacetic acid (TCA). Samples were centrifuged (10,000 rpm, 10 min.) and the supernatant was transferred to HPLC centrifilter for centrifiltration (10,000 rpm, 10 min). The mobile phase consisted of 0.1% acetic acid-acetonitrile (70:30, v/v, pH=3.2) solution at a flow rate of 0.5 ml/min for 3 minutes, followed by 0.1% acetic acid-acetonitrile (60:40, v/v) at a flow rate of 0.8 ml/min for 3 minutes. A stainless steel analytical column (Zorbax SB-C18, 2.1 mm ID×150.0 mm, 5 μm with a Phenomenex cartridge guard column) was used as the stationary phase. The column temperature was 50° C. Quantification of the erythromycylamine was performed using a HP 1100 LC/MSD API-Electrospray System. Data acquisition was set in the selective ion monitoring mode. The method was linear (r>0.9990) in the concentration range of 0.01 to 50 μg/ml. The absolute recovery was 95.0±2.19%.

Lung samples were homogenized with DI water. Oleandomycin was added to the samples as an internal standard. The homogenate was deproteinated with 0.9 M TCA. Samples were centrifuged at 10,000 rpm for 10 minutes and the supernatant was transferred to HPLC centrifilters for centrifiltration. The mobile phase consisted of 0.1% acetic acid-acetonitrile (70:30, v/v, pH=3.2) at a flow rate of 0.5 ml/min for 3 minutes, followed by 0.1% acetic acid-acetonitrile (60:40, v/v) at a flow rate of 0.8 ml/min for 3 minutes. A stainless steel analytical column (Zorbax SB-C18, 2.1 mm ID×150.0 mm, 5 μm with a Phenomenex cartridge guard column) was used as the stationary phase. The column temperature was 50° C. Quantification of the erythromycylamine was performed using a HP 1100 LC/MSD API-Electrospray System. Data acquisition was set in the selective ion monitoring mode. The linearity (r>0.9990) of the assay ranged from 0.1 to 200 μg/g. The extraction efficiency was 93.8±2.54%.

Pharmacokinetic parameters, area under the curve (AUC) and mean residence time (MRT), were estimated based on the statistical moment theory using WinNonlin™ Professional Version 2.0 software (Pharsight Corporation). The peak concentration (Cₘₐₓ) was not estimated but observed.

Inhalation Pharmacokinetics: For a 60 mg/mL solution, 3.191 g (4.08 mmol) of erythromycylamine (94% purity) was added to 43 mL of DI water and 4.27 mL (4.27 mmol) of 1 M sulfuric acid in a 50 ml volumetric flask. The solution was then adjusted to pH 6.5 with the addition of another 53 μL (0.053 mmol) of 1 M sulfuric acid. The volume was brought up to 50 mL with additional DI water. The 30 mg/mL solution was made by diluting the 60 mg/mL solution in ½ normal saline. The osmolality of the resulting solutions were 148 mOsm as determined using The Advanced™ Micro-Osmometer Model 3300 (Advanced Instruments, Inc., Norwood, Mass.)

Rats were exposed once to either 30 or 60 mg/mL solution of erythromycylamine sulfate via inhalation in a 32-port nose-only rodent exposure system (Battelle, Richland, Wash.) for 30 minutes. The Battelle system nose-only rodent exposure system is based on the Cannon Flow-Past Nose only system (Am. Ind Hyg Assoc J December, 1983; 44(12)923-8) and is made up of four stackable stain-less steel tiers with a total of 32 ports. The system includes inlet and exhaust flow monitoring and control, aerosol data was collected using the NORES version 1.1.4 software provided by Battelle. Erythromycylamine solutions were aerosolized using the PARI LC STAR™ nebulizer. Mean aerosol concentrations were determined by gravimetric analysis of filter samples taken at 10 and 20 minutes following the start of exposure. The mean aerosol concentrations were 0.54±0.06 and 1.36±0.30 mg/L, respectively, for 30 and 60 mg/mL solutions.

Lung and blood samples were collected from 3 rats at 0.083, 0.25, 0.5 1, 2, 4, 8 and 24 hours post dosing as described above. The sample collection and handling procedures for the inhalation study were same as for the intravenous study.

Bioanalytical assay procedures for the inhalation study were the same as for the intravenous study. The calculated deposited dose in the lung (pulmonary dose) was approximately 0.70 or 1.77 mg/kg following an inhalation dose of 30 or 60 mg/mL erythromycylamine solution for 30 minutes, respectively. The pulmonary dose in the lung was calculated as follows:

LDD=MAC×MV×DE×FLD÷MBW

Where,

LDD=Lung Deposited Dose

MAC=Mean Aerosol Concentration=0.54 and 1.36 mg/L for 30 and 60 mg/mL solutions, respectively.

MV=Minute Volume=0.1 L/min.

DE=Duration of Exposure=30 minutes

FLD=Fraction of Lung Deposit=0.1

MBW=Mean Body Weight=0.23 kg

Pharmacokinetic parameters in the lung following intravenous and inhalation administration of erythromycylamine are summarized below in Table 4:

TABLE 4
Pharmacokinetic parameters of erythromycylamine in the lung
following an intravenous or two inhalation doses in the rat (N = 3).

| | Drug Administration Route and Dose | | |
|---|---|---|---|
| | | Inhalation | Inhalation |
| | | 30 mg/mL, | 60 mg/mL, |
| Pharmacokinetic | Intravenous | (Pulmonary | (Pulmonary Dose: |
| Parameter | 25 mg/kg | Dose: 0.7 mg/kg) | 1.77 mg/kg) |
| Cₘₐₓ (μg/gram) | 68.99 | 89.33 | 155.24 |
| AUC (μg · h/gram)¹ | 605.19 | 854.27 | 1357.35 |
| AUC (μg · h/gram)² | 24.21 | 1220.39 | 766.86 |
| MRT(h)³ | 10.8 | 10.5 | 11.2 |

¹Area under the curve estimated 0-24 hours postdose.
²Area under the curve dose-normalized to 1 mg/kg.
³Mean residence time estimated 0-24 hours postdose.
n.e.: Not estimated.

### Example 7

### Aerosol and IV Efficacy of Erythromycylamine in the S. Pneumonia Rat Lung Model of Infection

Methods Male Sprague-Dawley rats were infected by intratracheal administration with 50-100 microliters of S. pneumoniae A66 (Strain #PGO 4716) prepared in agar beads. The inoculum was prepared by suspending a broth culture of PGO 4716 in molten agar, suspending the agar suspension in sterile mineral oil with mixing to generate small beads of agar containing the bacteria. The beads are recovered by centrifugation, resuspended in sterile saline, and administered to each animal through a tracheal incision by injection directly into the lung.

Erythromycylamine solutions are prepared in sterile saline. Antibiotic was administered either by intravenous injection into the tail vein or by aerosol exposure. The aerosol exposure was accomplished by nose-only exposure using the In-Tox Aerosol Exposure System (model No. 04-1100). This system is a closed aerosol delivery system designed to expose rodents that are confined in plastic tubes, open to the system at one end (nose port) and sealed at the other to maintain system integrity. The aerosol is generated by a Pari LC Star™ air-jet nebulizer at a flow of approximately 6.5 liters per minute. Vacuum is set at 9 liters per minute such that the total flow through the system with diluter air is 7.5 liters per minute.

Treatment is initiated 24 hours after infection and continued once per day, for 3 days. Aerosol was administered for 30 minutes each day. On day four after infection and 12 hours after the last dose, animals are sacrificed and lungs surgically removed. After removal, lungs are homogenized, diluted and quantitatively plated onto blood agar. Plates are incubated for 24 hours and colonies of S. pneumoniae counted to determine bacterial load. The results are shown in Table 5:

TABLE 5
Efficacy of Erythromycylamine vs. S. pneumoniae
in the Rat Pneumonia Model

| | Dose | CFU/gram |
|---|---|---|
| Route | (mg/kg per day) | Recovered |
| IV | 0 | 8.5 × 10⁷ |
| | 10 | BQL* |
| | 20 | BQL* |
| | 40 | BQL* |
| Aerosol | 0 | 4.1 × 10⁷ |
| | 0.13 | 3.5 × 10² |
| | 0.67 | BQL* |
| | 1.33 | BQL* |

*BQL = Below Quantitation Limit

### EXAMPLE 8

### Aerosol Efficacy of Erythromycylamine in the S. Pneumonia Rat Pulmonary Model of Infection After a Single Dose Treatment of Erythromycylamine

Male Sprague-Dawley rats were infected and exposed to aerosol treatment as described in Example 7. The single treatment was initiated 24 hours after infection with aerosol administered at the doses indicated for 30 minutes. No further treatment was undertaken and animals were observed until surgery. On day four after infection (day 3 after dosing), the animals were sacrificed and their lungs were surgically removed. After removal, the lungs are homogenized, diluted and quantitatively plated onto blood agar. The plates are incubated for 24 hours and colonies of S. pneumoniae are counted to determine bacteria load. The results after a single dose administration are shown in FIG. 11. Further results are shown in Table 6:

TABLE 6
Efficacy of Erythromycylamine vs. S. pneumoniae
in the Rat Pneumonia Model

| Number | Dose | CFU/gram |
|---|---|---|
| of Doses | (mg/kg/day) | Recovered |
| 3 | 0 | 4.1 × 10⁷ |
| 3 | 0.13 | 3.5 × 10⁷ |
| 3 | 0.67 | BQL |
| 3 | 1.33 | BQL |

BQL = below quantitation limit

### EXAMPLE 9

### Aerosol Delivery of Erythromycylamine to Dogs Characterization of Aerosol Pharmacokinetics

Inhalation Pharmacokinetics: For a 60 mg/mL solution, 3.191 g (4.08 mmol) of erythromycylamine (94% purity) was added to 43 mL of DI water and 4.27 mL (4.27 mmol) of 1 M sulfuric acid in a 50 ml volumetric flask. The solution was then adjusted to pH 6.5 with the addition of another 53 μL (0.053 mmol) of 1 M sulfuric acid. The volume was brought up to 50 mL with additional DI water. Dogs were exposed once to either 60 mg/mL solution of erythromycylamine sulfate via an inhalation mask exposure system (Inveresk Research, Scotland, UK) for 30 minutes.

The dogs were removed from their pen in the dog holding area and transferred to the dosing laboratory. During dosing the animals were either restrained by an animal attendant or in a sling/harness system. Inhalation dosing was undertaken using a closed facemask connected to a nebulizer that was suitably characterized prior to commencement of dosing. The dosing apparatus incorporates a facemask and mouthpiece attached to flexible tubing, which was connected to the nebulizer device. The mouthpiece was located inside the animal's mouth, on top of the tongue, and the facemask sealed around the dog's snout by means of a rubber sleeve. An exhaust valve from the mask was connected to an extract system. When the dosing apparatus is fully assembled and fitted to the dog, inspiration is shown by movement of the aerosol through the flexible tubing to the dog.

Lung samples were collected from 2 dogs at 2, 24, 48, 72, 96 and 120 hours post-dosing. Lungs were removed surgically from the dogs, and each lobe (right caudal, left caudal, right cranial, left cranial, right middle and accessory). was separated for assay. Plasma samples were collected from all surviving animals at 2, 24, 48, 72, 96 and 120 hours post.

Erythromycylamine concentrations in plasma and the lung (per gram of lung tissue) were determined using a LC-MS method. Plasma samples (100 μg) were spiked with oleandomycin (internal standard, 1 μg/mL) before extraction. Plasma samples (100 μL) were deproteinated with 3.3% trichloroacetic acid (TCA). Samples were centrifuged (10,000 rpm, 10 min.) and the supernatant was transferred to HPLC centrifilter for centrifiltration (10,000 rpm, 10 min). The mobile phase consisted of 0.1% acetic acid-acetonitrile (70:30, v/v, pH=3.2) solution at a flow rate of 0.5 ml/min for 3 minutes, followed by 0.1% acetic acid-acetonitrile (60:40, v/v) at a flow rate of 0.8 ml/min for 3 minutes. A stainless steel analytical column (Zorbax SB-C18, 2.1 mm ID×150.0 mm, 5 μm with a Phenomenex cartridge guard column) was used as the stationary phase. The column temperature was 50° C. Quantification of erythromycylamine was performed using a HP 1100 LC/MSD API-Electrospray System. Data acquisition was set in the selective ion monitoring mode. The method was linear (r>0.9990) in the concentration range of 0.01 to 50 μg/ml. The absolute recovery was greater than 90%.

Lung samples were homogenized with DI water. Oleandomycin was added to the samples as an internal standard. The homogenate was deproteinated with 0.9 M TCA. Samples were centrifuged at 10,000 rpm for 10 minutes and the supernatant was transferred to HPLC centrifilters for centrifiltration. The mobile phase consisted of 0.1% acetic acid-acetonitrile (70:30, v/v, pH=3.2) at a flow rate of 0.5 ml/min for 3 minutes, followed by 0.1% acetic acid-acetonitrile (60:40, v/v) at a flow rate of 0.8 ml/min for 3 minutes. A stainless steel analytical column (Zorbax SB-C18, 2.1 mm ID×150.0 mm, 5 μm with a Phenomenex cartridge guard column) was used as the stationary phase. The column temperature was 50° C. Quantification of the erythromycylamine was performed using a HP 1100 LC/MSD API-Electrospray System. Data acquisition was set in the selective ion monitoring mode. The linearity (r>0.99) of the assay ranged from 2 to 100 μg/g for lung. The extraction efficiency was greater than 90%.

Pharmacokinetic parameters, area under the curve (AUC) and mean residence time (MRT) and half-life (T_{1/2}) were estimated based on the statistical moment theory using WinNonlin™ Professional Version 3.1 software (Pharsight Corporation). The peak concentration (Cₘₐₓ) was not estimated but observed.

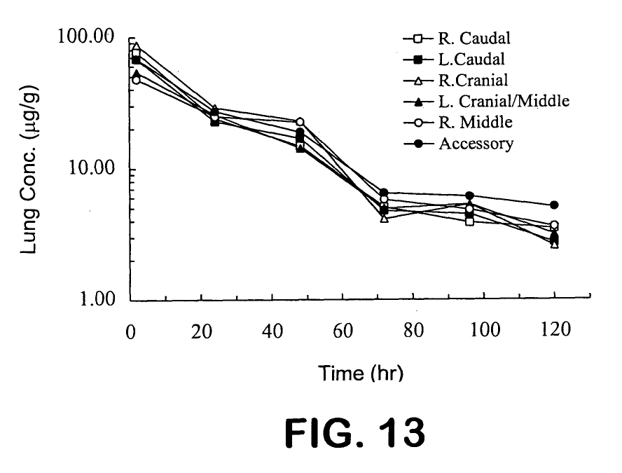

Pharmacokinetic parameters in the lung and plasma inhalation following administration of erythromycylamine in the dog are summarized in Table 7 below, and in FIGS. 13 and 14:

TABLE 7
Pharmacokinetic Parameters of Erythromycylamine in
the Lung and Plasma following a 30-minutes Inhalation
Administration of 60 mg/mL Solution in the Dog (N = 2).

| | Pharmacokinetic Parameter (unit) | | | |
|---|---|---|---|---|
| | Cₘₐₓ | AUC(0-120 h) | | |
| Matrix | (μg/gram) | (μg · h/gram)¹ | t_{1/2} (h) | MRT (h)¹ |
| Whole Lung | 69 | 2085 | 27 | 29 |
| Plasma | 1.0 | 29 | n.e. | 37 |
| Right Caudal | 77 | 2078 | 26 | 26 |
| Left Caudal | 68 | 2000 | 26 | 28 |
| Right Cranial | 87 | 2517 | 24 | 27 |
| Left Cranial | 54 | 1857 | 29 | 31 |
| Right Middle | 48 | 1979 | 30 | 34 |
| Accessory | 68 | 2256 | 31 | 31 |

¹Mean residence time
n.e.: Not estimated.

### EXAMPLE 10

### Liquid Aerosol Delivery of Erythromycylamine

A solution of erythromycylamine sulfate (100 mg/mL) in quarter normal saline at pH 7.0 is prepared in accordance with the general procedure of the foregoing examples. A 1.0 mL dose of the solution is administered by aerosol inhalation in less than 10 minutes to a human subject suffering from acute exacerbation of chronic bronchitis (AECB) using an AeroGen Aerodose™ inhaler. A reduction in the bacteria associated with AECB and symptoms of AECB is observed.

### EXAMPLE 10

### Dry Powder Aerosol Delivery of Erythromycylamine

A dry powder formulation of erythromycylamine sulfate (100 mg) and a dry powder carrier (equal parts of lactose, 2-hydroxypropyl-β-cyclodextrin, mannitol and aspartame; total weight 25 mg) is prepared. The formulation is administered by aerosol inhalation in less than 2 minutes to a human subject suffering from acute exacerbation of chronic bronchitis (AECB) using a Glaxo Ventolin Rotohale™ inhaler. A reduction in the bacteria associated with AECB and symptoms of AECB is observed.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. An aerosol formulation for inhibition of susceptible bacteria in the endobronchial space of a subject suffering from an endobronchial infection,
said formulation comprising from about 50 mg to about 750 mg of a macrolide antibiotic and a pharmaceutically acceptable carrier capable of being administered in aerosol form using a jet nebulizer, a ultrasonic nebulizer, a vibrating porous plate nebulizer or a dry powder inhaler able to produce aerosol particles having a mass median aerodynamic diameter between 1 and 5 μm in size.

2. The aerosol formulation of claim 1 wherein the macrolide antibiotic is selected from the group consisting of erythromycylamine, dirithromycin, erythromycin A, clarithromycin, azithromycin, and roxithromycin.

3. The aerosol formulation of claim 1 wherein the macrolide antibiotic is erythromycylamine.

4. The aerosol formulation of claim 1 having a pH is in the range of 5.0 to 7.0.

5. The aerosol formulation of claim 1 wherein the nebulizer is jet nebulizer.

6. The aerosol formulation of claim 1 wherein the nebulizer is an ultrasonic nebulizer.

7. The aerosol formulation of claim 1 wherein the nebulizer is a vibrating porous plate nebulizer.

8. The aerosol formulation of claim 1 wherein the susceptible bacteria are selected from the group consisting of Streptococcus pneumoniae, Haemophilus influenzae, Staphylococcus aureus, Moraxella catarrhalis, Legionella pneumonia, Chlamydia pneumoniae, and Mycoplasma pneumoniae.

9. The aerosol of claim 8 wherein the pH is 6.0.

10. The aerosol of claim 9 wherein the nebulizer is a jet nebulizer.

11. The aerosol of claim 9 wherein the nebulizer is an ultrasonic nebulizer.

12. The aerosol of claim 9 wherein the nebulizer is a vibrating porous plate nebulizer.

13. A method for treatment of susceptible bacterial endobronchial infections by administering to a subject in need of such treatment an aerosol formulation for inhalation comprising about 50 mg to about 750 mg of a macrolide antibiotic and a pharmaceutically acceptable carrier capable of being administered in aerosol form using a jet nebulizer, an ultrasonic nebulizer, a vibrating porous plate nebulizer or a dry powder inhaler able to produce aerosol particles having a mass median aerodynamic diameter between 1 and 5 μm in size.

14. The method of claim 13 wherein the macrolide antibiotic is selected from the group consisting of erythromycylamine, dirithromycin, erythromycin A, clarithromycin, azithromycin, and roxithromycin.

15. The method of claim 13 wherein the macrolide antibiotic is erythromycylamine.

16. The method of claim 13 wherein the pH of the aerosol formulation is in the range of 5.0 to 7.0.

17. The method of claim 13 wherein the nebulizer used for administration of the aerosol formulation is a jet nebulizer.

18. The method of claim 13 wherein the nebulizer used for administration of the aerosol formulation is a ultrasonic nebulizer.

19. The method of claim 13 wherein the nebulizer used for administration of the aerosol formulation is a vibrating porous plate nebulizer.

20. The method of claim 13 wherein the susceptible bacteria are selected from the group consisting of Streptococcus pneumoniae, Haemophilus influenzae, Staphylococcus aureus, Moraxella catarrhalis Legionella pneumonia, Chlamydia pneumoniae, and Mycoplasma pneumoniae.

21. The method of claim 13 wherein a dose of less than about 2.0 ml of a nebulized liquid aerosol formulation comprising form about 50 to about 150 mg/ml of the macrolide antibiotic is administered to the subject in less than about 10 minutes.

22. The method of claim 21 wherein the dose comprises less than about 1.5 ml of the nebulized aerosol formulation.

23. The method of claim 21 wherein the dose comprises less than about 1.0 ml of the nebulized aerosol formulation.

24. The method of claim 20 wherein the aerosol formulation comprises from about 70 to about 130 mg/ml of the macrolide antibiotic.

25. A unit dose device, comprising a container containing less than about 2.0 ml of a macrolide antibiotic formulation comprising from about 50 to about 150 mg/ml of a macrolide antibiotic in a liquid physiologically acceptable carrier.

26. A unit dose device of claim 25 which contains less than about 1.5 ml of the macrolide antibiotic formulation.

27. A unit dose device of claim 25 which contains less than about 1.0 ml of the macrolide antibiotic formulation.

28. A unit dose device of claim 25 wherein the macrolide antibiotic formulation comprises from about 70 to about 130 mg/ml of the macrolide antibiotic.

29. A unit dose device of claim 25 wherein the macrolide antibiotic formulation comprises from about 90 to about 110 mg/ml of the macrolide antibiotic.

30. A unit dose formulation of claim 25 wherein the macrolide antibiotic is selected from the group consisting of erythromycylamine, dirithromycin, erythromycin A, clarithromycin, azithromycin, and roxithromycin.

31. The method of claim 25 wherein the macrolide antibiotic is erythromycylamine.

32. A unit dose device of claim 25 which contains less than about 2.0 ml of a macrolide antibiotic formulation comprising from about 20 to about 200 mg/ml of erythromycylamine.

33. A unit dose device, comprising a container containing a macrolide antibiotic formulation comprising from about 25 to about 250 mg of a macrolide antibiotic in a dry powder physiologically acceptable carrier.

34. A unit dose device of claim 33 wherein the macrolide antibiotic formulation comprises from about 50 to about 200 mg of the macrolide antibiotic.

35. A unit dose device of claim 33 wherein the macrolide antibiotic formulation comprises from about 75 to about 150 mg of the macrolide antibiotic.

36. A unit dose device of claim 33 wherein the macrolide antibiotic is selected from the group consisting of erythromycylamine, dirithromycin, erythromycin A, clarithromycin, azithromycin, and roxithromycin.

37. A unit dose device of claim 33 wherein the macrolide antibiotic is erythromycylamine.

38. A unit dose device of claim 33 wherein the macrolide antibiotic formulation comprises from about 50% to about 90% by weight of the macrolide antibiotic.
